(19) United States
(12) Patent Application Publication
(10) Pub. No.: US 2007/0270864 A1
(43) Pub. Date: Nov. 22, 2007

(54) INTRAMEDULLARY TRANSILLUMINATION APPARATUS, SURGICAL KIT AND METHOD FOR ACCURATE PLACEMENT OF LOCKING SCREWS IN LONG BONE INTRAMEDULLARY RODDING

(76) Inventor: James P. Gurtowski, Huntington, NY (US)

Correspondence Address: David Aker, 23 Southern Road, Hartsdale, NY, 10530, US

(21) Appl. No.: 11/744,844

(22) Filed: May 5, 2007

Related U.S. Application Data
(60) Provisional application No. 60/797,986, filed on May 5, 2006.

(51) Int. Cl. A61B 17/00

(52) U.S. Cl. 606/79

(57) ABSTRACT

Apparatus, including a surgical kit, for use with a surgical drill, in the repair of bones using an intramedullary nail insertable into a patient's bone, comprising a rod like device for insertion into the intramedullary nail, the device having a light source emitting electromagnetic non-ionizing radiation in the infrared or visible portions of the electromagnetic spectrum, and the device being positionable so that the light source emits the radiation through a distal transverse hole of the intramedullary nail; and a surgical instrument for exposing an exterior surface of a portion of the bone illuminated by the radiation for view by the surgeon. The surgeon can detect the radiation on the exterior surface of the bone and align the surgical drill to the radiation passing through the transverse hole of the intramedullary nail, permitting accurate drilling of a hole through the bone and passage of the drill through the transverse hole of the intramedullary nail.

This application claims priority, under 35 U.S.C. §119(e), from provisional patent application Ser. No. 60/797,986 filed on May 5, 2006, which is hereby incorporated herein, by reference, in its entirety.

### BACKGROUND OF THE INVENTION

1. Field of the Invention

The present invention relates to the apparatus, methods, technique and accuracy of the placement of distal locking screws during the procedure of long bone intramedullary nailing (also known as: intramedullary rodding and for the purposes of this description will be used synonomously). More particularly, it relates to those apparatus, methods and systems for accurate placement of distal locking screws.

2. Background Art

Generally since the introduction of the locked nail for intramedullary fixation of long bones, ie. humerus, radius, ulna, femur, tibia the technique of screw fixation for distal interlock has become accepted. This procedure is performed with the use of intraoperative x-ray, or continuous fluoroscopy. Most currently available products available for intrameduallary rodding describe surgical techniques requiring biplane x-ray for placement of distal locking screws. There have been attempts at modifying the technique of distal locking to other types of fixation such as fins or blocking devices but these have proved less reliable.

Distal locking of intrameduallary devices is subject to numerous complications, including malplacement of the screws, multiple attempts at passing the screws, nerve, and surrounding tissue damage and missing the interlock hole of the nail or intramedullary device.

The placement of the distal locking screws is time consuming increasing the operative time, increases the radiation exposure to the patient, increases the radiation exposure to the operating room staff and the surgeon.

FIGS. 1A and 1B illustrate a typical long bone, such as a bone 20. As illustrated in FIG. 2A and FIG. 2B, using conventional techniques, a hole 22 is drilled, starting at the top of a bone 20, such as a femur. An intrameduallary guide wire 24 having a reamer 16 at its end is worked down the bone 20 to clear the canal 28. An intrameduallary rod (generally visible only with use of x-ray techniques) having screw holes is inserted into the broken bone 20, at the proximal end. External fixtures or aiming arms can be used to properly place screws at the proximal end of the bone 20. However, in accordance with conventional techniques, once the limb of the patient has been manipulated into the precise position in which the fracture is to heal, it is necessary to use a continuous x-ray technique, such as fluoroscopy, to align an appropriate drill, with the holes in the distal end of the bone 20, to drill appropriate holes, for distal end locking screws. As described above, this is extremely difficult to accomplish with great precision, and the consequences of misplacement can be rather dire with respect to the proper relative positioning of the portions of the fractured bone 20, and possible nerve, and surrounding tissue damage.

U.S. Pat. No. 5,417,688 to John A. Elstrom et al. illustrates a well though out approach for solving this problem. As indicated in its abstract this patent is directed to: Apparatus and methods for detecting the location of transverse holes of an intramedullary nail which has been inserted into a long bone and for aligning a surgical drill to the transverse holes. An insert having a light source at its distal end emitting non-ionizing electromagnetic radiation in the visible spectrum or infrared is inserted into the intramedullary nail, such that the light source is placed adjacent to the transverse holes. The light source may be a directional radiation output emitting the radiation in a direction perpendicular to the axis of the intramedullary nail. The light source may alternatively be an isotropic radiation output emitting radiation in all directions. The surgeon detects the transmitted radiation on the surface of the body of the patient and aligns the drill with the emitted radiation.

FIG. 3 herein is essentially a reproduction of FIG. 1 of U.S. Pat. No. 5,417,688, while FIG. 4 herein is essentially a reproduction of FIG. 10 of U.S. Pat. No. 5,417,688. Incorporated by reference herein in their entireties are the teachings of this patent, as well as those of U.S. Pat. No. 5,540,691, also issued to Elstrom et al. (on an application that was a continuation-in-part of the application that issued as U.S. Pat. No. 5,417,688), which teaches that auxiliary video equipment, such as a camera and video monitor may be used to collect and display the emitted radiation, particularly if light in the infrared portion of the spectrum is used.

### SUMMARY OF THE INVENTION

As far as is known, the techniques and apparatus of the abovementioned Elstrom et al. patents have not been commercially developed. One possible reason for this may be that while the concept is sound, there are practical difficulties in aligning the drill based on the light or infrared energy that reaches the surface or skin of the patient. It has been found that the light energy is diffused by travel from the bone being repaired to the surface of the skin. While this may not pose serious difficulties in the case of portions of bones located very close to the surface of the skin (especially if the patient does not carry excess tissues due to obesity), in many cases, the travel through the patient's tissues, from the bone to the skin, tends to diffuse the energy, (which is a well defined circle on the surface of the bone) into an ill defined illuminated region, which, in some cases, and in some patients, is of relatively little assistance in providing guidance to the surgeon in the placement of locking screws.

It is a first object of the present invention to provide apparatus and surgical instruments to improve the accuracy and precision of drilling holes in bones for locking screws for intramedullary nails.

It is an object of the invention to provide a device which will pinpoint the interlocking hole of the intramedullary nail.

It is a further object of the invention to provide this technique minimizing radiation exposure to the patient and operative staff.

It is another object of the invention to function with most currently available devices for intramedullary nailing without significant modification.

It is an additional object of the device to function with generally available arthroscopic light sources.

It is yet another object of the invention to provide a method and apparatus which facilitate treatment of long bone fractures under less than ideal treatment facilities, such as military field hospitals, or when reliable x-ray equipment may not be available.

These objects and others are achieved in accordance with the invention by:

1. Providing a flexible fiberoptic or similar device which once the intramedullary rod is in position can be inserted in the rod's existing canullation.

2. Providing a suitable connection to a high intensity light source (available arthroscopic or suitable light source)

3. Providing a flexible device of suitable light transmitting capability to illuminate the silhouette of the intramedullary rod distal locking hole.

4. Providing a radiographic marker to verify position with existing x-ray equipment.

5. Providing suitable calibration for length of insertion into the intrameduallary device.

6 Providing the device in various diameters, lengths and sizes to facilitate use with various size requirements of existing and future manufactured canulated devices.

7. Providing a described technique for use including but not limited to insertion, localization, and endoscopic distal locking based on visualization of the transillumination of the distal locking hole.

8. Providing techniques, using cannulated instruments, for placement of locking screws after proper position has been determined.

Thus, the invention is directed to an apparatus, for use (by a surgeon or surgical robot) with a surgical drill in the repair of bones using an intramedullary nail insertable into a patient's bone, the intramedullary nail having a hollow body portion and a distal transverse hole. The apparatus comprises in combination a rod like device for insertion into the intramedullary nail, the device having a light source emitting electromagnetic non-ionizing radiation in the infrared or visible portions of the electromagnetic spectrum, and the device being positionable so that the light source emits the radiation through the distal transverse hole of the intramedullary nail; and a surgical instrument for exposing an exterior surface of a portion of the bone illuminated by the radiation for view by the surgeon; whereby visual detection of the radiation on the exterior surface of the bone (directly by the surgeon or by a camera) and alignment of the surgical drill to the radiation passing through the transverse hole of the intramedullary nail aligns the surgical drill with the transverse hole, permitting accurate drilling of a hole through the bone and passage of the drill through the transverse hole of the intramedullary nail.

The surgical instrument may comprise a primary cannula configured for extending from outside the patient to a location within the patient from which the portion of the bone illuminated by the radiation is visible; and an arthroscope cannula for insertion into the primary cannula to allow the surgeon to view the exterior surface of the portion of the bone illuminated by the radiation.

The surgical instrument may comprise a primary cannula configured for extending from outside the patient to a location within the patient from which the portion of the bone illuminated by the radiation is visible; a guide pin cannula for insertion within the primary cannula; and a threaded guide pin for insertion into the guide pin cannula, and for making an initial opening in the portion of the bone illuminated by the radiation. The surgical instrument may further comprising a cannulated drill having a cannula sized to accept the guide pin so that the drill is guided by the guide pin to drill the hole through the bone and to assist in aligning of the drill so that the drill passes through the transverse hole of the intramedullary nail, when the device is moved within the intramedullary nail so that the device is clear of the transverse hole, and the guide pin cannula has been removed from the primary cannula.

The surgical instrument may comprise a screw driver for insertion into the primary cannula, for driving a screw into the bone and through the transverse hole of the intramedullary nail.

The surgical instrument may comprise a scalpel for making an incision in tissues of the patient to permit the surgeon to directly view the exterior surface of a portion of the bone illuminated by the radiation. The surgical instrument may further comprise a retractor for retracting the tissues of the patient on at least one side of the incision, to assist the surgeon in obtaining the direct view of the exterior surface of a portion of the bone illuminated by the radiation.

The combination may further comprise the surgical drill for drilling the hole through the bone and passing through the transverse hole of the intramedullary nail.

The light source may comprise a laser. The apparatus may comprise a shaping mask for the light source for providing a shape to a beam of radiation from the device, to assist in precisely locating a center of the beam.

The invention is also directed to a surgical kit container apparatus for performing a surgical procedure as described herein, and to the method for performing the surgical procedure.

### BRIEF DESCRIPTION OF THE DRAWINGS

The foregoing aspects and other features of the present invention are explained in the following description, taken in connection with the accompanying drawings, wherein:

FIG. 1A is a plan view that represents a typical long bone, such as the femur.

FIG. 1B represent the bone of FIG. 1A, as viewed from a different angle.

FIG. 2A is a plan view, in partial cross-section, showing an intrameduallary guide wire and typical reamer for opening a canal prior to intrameduallary rod insertion into the bone of FIG. 1A and FIG. 1B.

FIG. 2B is an enlarged portion of FIG. 2A. FIG. 3 is taken from FIG. 1 of U.S. Pat. No. 5,417,688 to Elstrom et al.

FIG. 4 is taken from FIG. 10 of U.S. Pat. No. 5,417,688 to Elstrom et al.

FIG. 5 is a cross sectional view of an intramedullary illumination apparatus for use with the present invention.

FIG. 6 is a side elevational view and a top plan view of a portion of the apparatus of FIG. 5.

FIG. 7 is a plan view of a system, for use in accordance with the invention, for intramedullary illumination.

FIG. 8 illustrates a surgically exposed portion of a long bone with the apparatus of FIG. 5 and FIG. 7 in place.

FIG. 9 illustrates a first portion of a method, in accordance with the invention, for the placement of locking screws.

FIG. 10 illustrates a second portion of the method, in accordance with the invention.

FIG. 11 illustrates a third portion of the method, in accordance with the invention.

FIG. 12 illustrates a first step in the use of cannulated devices in accordance the invention, to assist in locking screw placement.

FIG. 13 illustrates a second step in the use of cannulated devices in accordance the invention, to assist in locking screw placement.

FIG. 14 illustrates a third step in the use of cannulated devices in accordance the invention, to assist in locking screw placement.

FIG. 15 illustrates a fourth step in the use of cannulated devices in accordance the invention, to assist in locking screw placement.

FIG. 16 illustrates a fifth step in the use of cannulated devices in accordance the invention, to assist in locking screw placement.

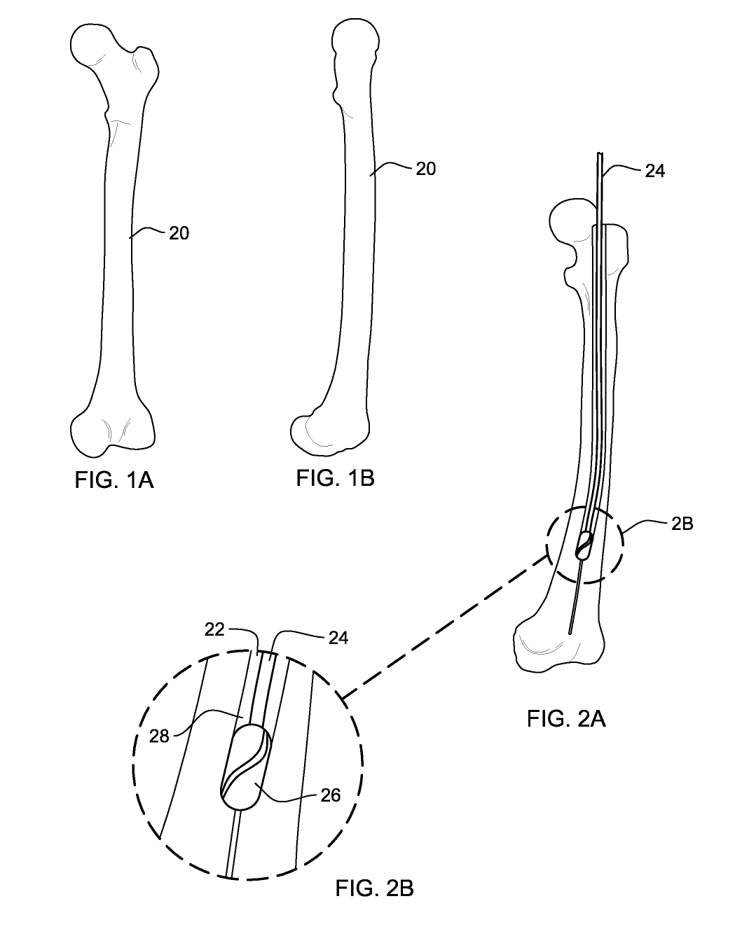

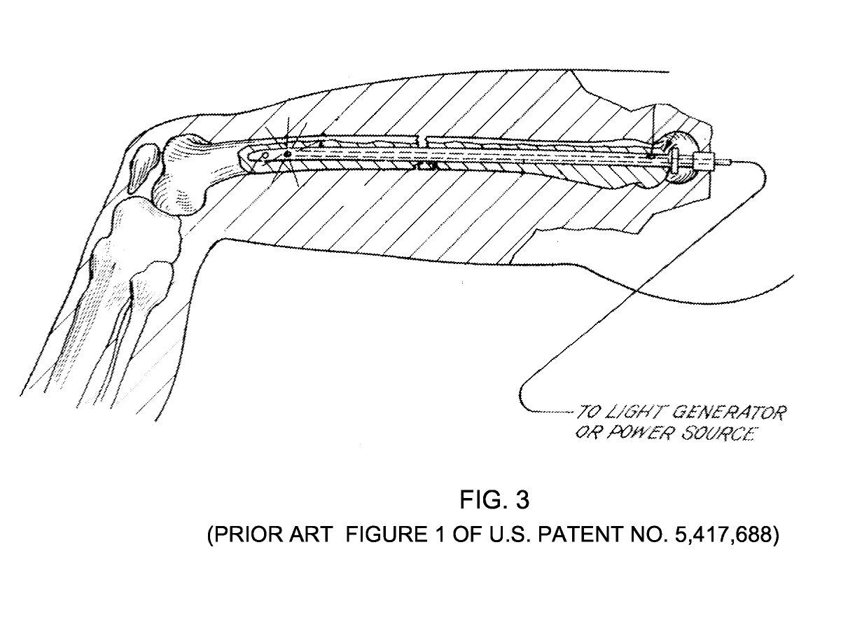

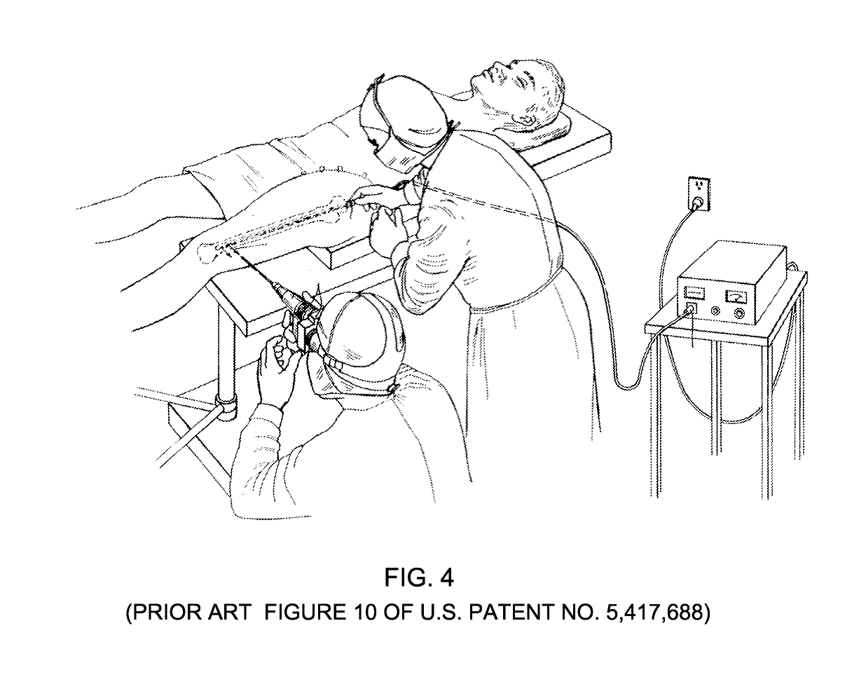

### DETAILED DESCRIPTION OF THE PREFERRED EMBODIMENTS

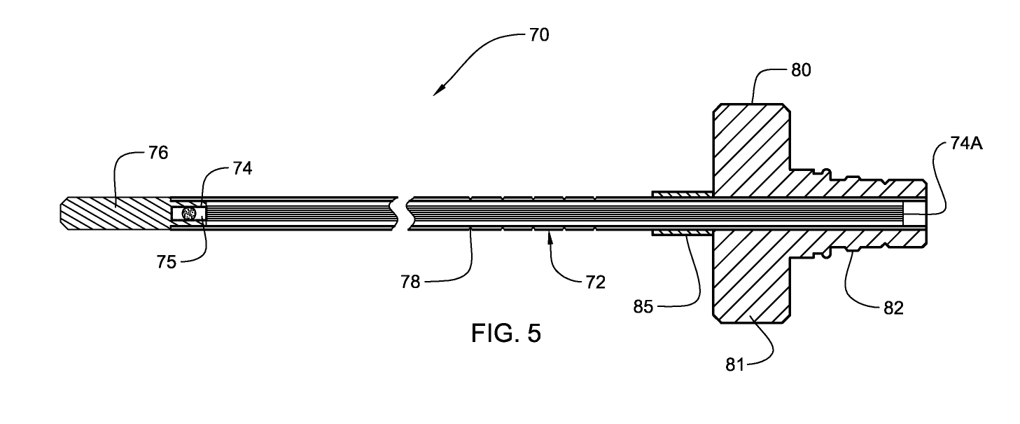

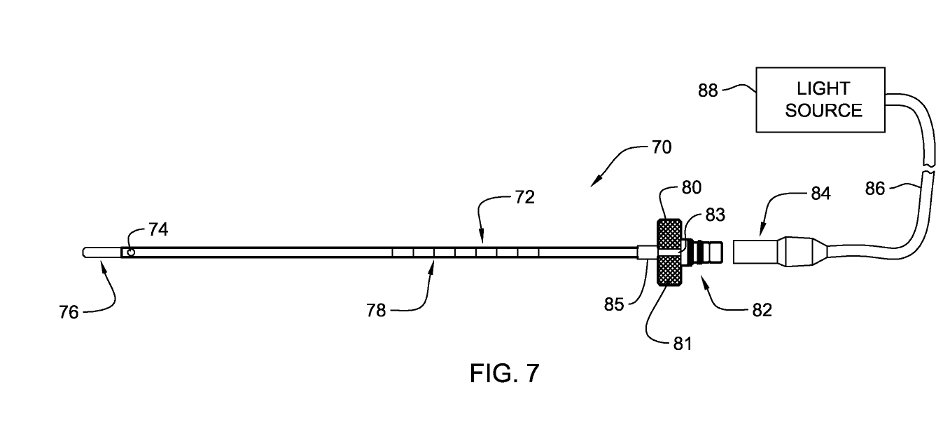

Reference is made to FIG. 5 for a cross-sectional view, and to FIG. 7, for a plan view of an apparatus 70 incorporating features of the present invention. Although the present invention will be described with reference to the embodiments shown in the drawings, it should be understood that the present invention can be embodied in many alternate forms of embodiments. In addition, any suitable size, shape or type of elements or materials could be used.

The apparatus 70 comprises a suitable light conduction device or member 72 for insertion into intrameduallary rod 30, generally after intrameduallary rod 30 is positioned in canal 28 of the bone 20 or other bone. Member 72 is designed to be somewhat flexible, and has a diameter permitting relatively easy insertion into intrameduallary rod 30. Thus, members 72 may be produced in several different outer diameters including but not limited to 2 mm, 2.5 mm, 3 mm, 3.5 mm 4 mm, 4.5 mm as necessary to correspond to existing cannulation of intramedullary rods in which guide wires are inserted; i.e. to match the inner diameters of various respective intrameduallary rods 30. Member 72 can be of suitable length based on the choice of use for various lengths of intramedullary devices or rods 30 suitable for various lengths for different bones, such as, for example humerus, radius, ulna, femur tibia.

Member 72 can have either a single fiber or preferably bundled fibers 73 with the ability, provided as explained below, to have light exit in a generally radial direction perpendicular to the length of member 72 at at least one optically transparent window 74 to pinpoint or variously as necessary to transilluminate the distal locking hole of an intramedullary rod 30. The individual fibers of bundled fibers 73 are bent at their distal ends by approximately ninety degrees, polished, and secured in a biocompatible, clear epoxy (not shown) so that the ends thus secured, and the epoxy in which they are embedded, close off an opening in member 72, thus defining an optically transparent window 74, which appears to be generally rectangular in shape when viewed from a direction perpendicular to the longitudinal axis of 72. While a window of this shape may be used, an optical beam shaping mask 75 may be provided to shape or pattern the beam of radiation from apparatus 70, to assist in precisely locating a center of the beam. While the opening therein is shown as a circle, it will be understood that various other patterns, such as cross hairs, may be used if the particular application does not unduly diffuse the light.

In a similar manner to the distal end, at their proximal end, the individual fibers of bundled fibers 73 are polished, and secured in the biocompatible, clear epoxy (not shown) so that the ends thus secured, and the epoxy in which they are embedded, close off the proximal end of member 72, thus defining an optically transparent window 74A, into which light may be transmitted.

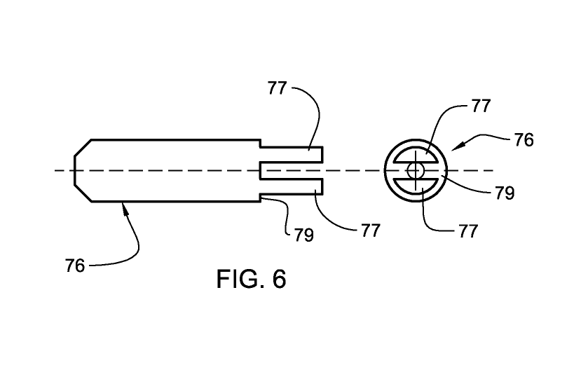

Member 72 may be constructed with a radioopaque portion or plug 76 (FIG. 6), as for example, at its distal end, to permit x-ray verification of its position in an intramedullary rod 30, when in place in the patient's bone. Plug 76 is advantageously designed to close off the distal end of member 72 when two prongs 77, integrally formed with plug 76 are tightly received within the tube which forms member 72. Thus, member 72 is not only sealed, by applying a suitable biocompatible adhesive to prongs 77 and to shoulder 79 of plug 76, before plug 76 is inserted into the end of member 72, but also has a suitable radiographic marker or markers including but not limited to distal radioopaque marker for verification of placement of the device. Further, a series of graduated markers, such as a series of markers 78 (FIG. 7) visible on the external surface of member 72, may also assist in determining the depth of insertion of member 72 into intramedullary rod 30. Thus, these graduations, or calibrations are used to determine depth of insertion as variously necessary for the use of the device.

A support handle or knob 80, knurled about its periphery 81, and into which member 72 extends and to which it is secured by a suitable biocompatible adhesive, may have a connector or be threaded 82 at its end to receive and mate with a corresponding connector 84 of a light source cable 86. A marker or notch 83 (FIG. 7), extending parallel to member 72, is provided on the periphery 81 of knob 80, and located to be in line with window 74, to assist rotational orientation of member 72 within a rod 30.

A tightly fitting reinforcing sleeve 85 is adhered about member 72 to prevent bending of member 72 near the point of entry into knob 80.

The end of cable 86 not coupled to apparatus 70, is connected to one of various types of light sources 88 of current a manufacture or future manufacture, including but not limited to generally available arthroscopic light sources variously manufactured under the names ACMI, Olympus, Storz, Wolf, Dyonics and others.

Technique for Use of Intramedullary Transillumination Apparatus:

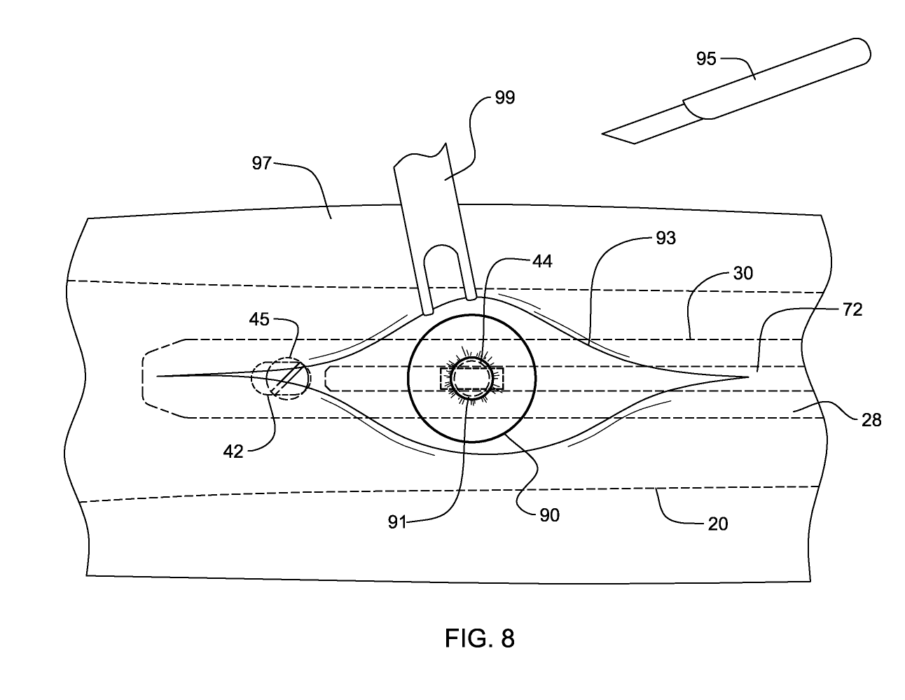

Referring to FIG. 8, following insertion of the cannulated intramedullary nail or rod 30 over a conventional guide wire (not shown) placed in the canal 28, the guide wire is removed and the member 72 of device for intramedullary transillumination is inserted. Member 72 is selected to be of appropriate size, diameter and length. Connector 84 of light source cable 86 is then attached and a suitably available light source 88 (FIG. 7) which provides sufficient luminosity of visible light, or a portion of the spectrum thereof, to transilluminate the medullary cortex and silhouette the distal locking hole of the intramedullary rod 30, is turned on. When member 72 is properly placed within intramedullary rod 30, light from optically transparent window 74 thus illuminates a distal locking hole of intramedullary rod 30. This creates a corona or silhouette 90 of a concentric hole visible external to the bone either by direct visualization through an incision 93 with a scalpel 95 (the tissues 97 of the patient being held back by a retractor 99) or through endoscopic cannulation over the site. The intensity of light from light source 88 is generally adjustable, and at an intensity setting that can be easily and rapidly determined by simple observation, the location of the locking hole will present a bright, often generally yellow center 91, surrounded by a generally red corona or silhouette 90 of diffused light. This permits precise determination of the position of the locking screw, even though the rod can not be seen, without the use of x-ray apparatus. It will be understood that using the procedures discussed above or below, a screw 45 is first placed in the most distal hole 42, and then in the next most distal hole 44, so that the member 72 may be withdrawn somewhat and is not damaged when a hole for screw 45 is drilled, and screw 45 is put in place.

Once the visible silhouette 90 of the hole is visualized, a conventional guide pin (not shown) can be introduced to mark the cortex of the bone 20. Member 72 of the apparatus 70 for intramedullary transillumination can then be removed from the patient and the guide pin can then be passed through the previously illuminated hole in intramedullary rod 30 and verified on x-ray for position.

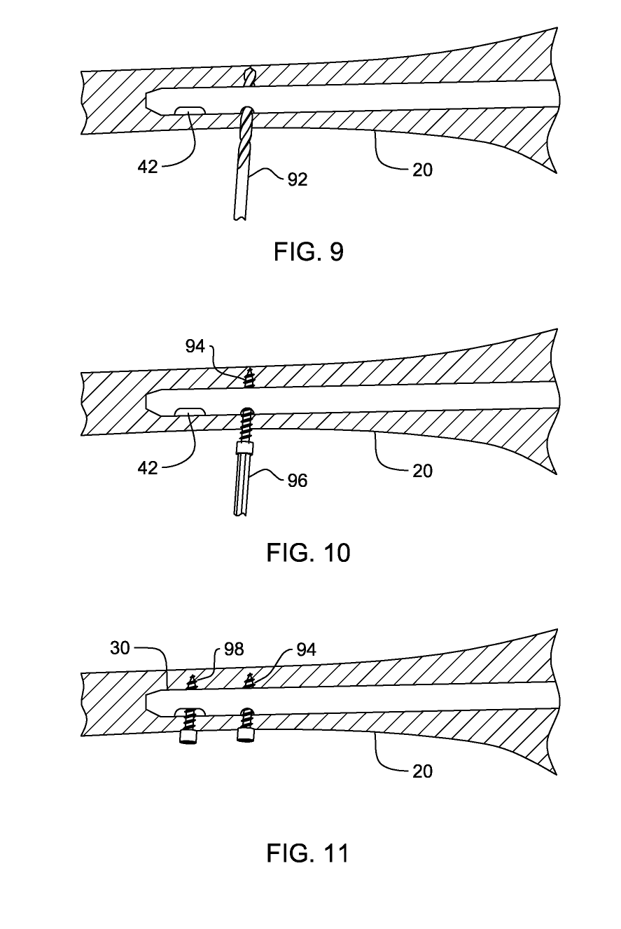

Referring to FIG. 9, FIG. 10 and FIG. 11, then using generally accepted technique, the procedure of distal locking can be completed, after using the technique and apparatus described above to achieve highly accurate positioning. In FIG. 9 a hole in the bone 20 is drilled using a conventional drill 92. In FIG. 10, a locking screw 94 is placed in hole 44 using a conventional insertion instrument 96. In FIG. 11, a locking screw 98 has been placed in hole 42 of rod 30 by the same technique. It will be understood that generally the procedure using the apparatus of the invention would be used first for locking screw 98, so that it could be inserted before locking screw 94. Otherwise the insertion of locking screw 94 would prevent the insertion of member 72 to a depth sufficient to illuminate the more distal hole 42.

Various cannulas can be developed to alternately accommodate the arthroscope, guide pin and ultimately the diameter of the final distal locking screws.

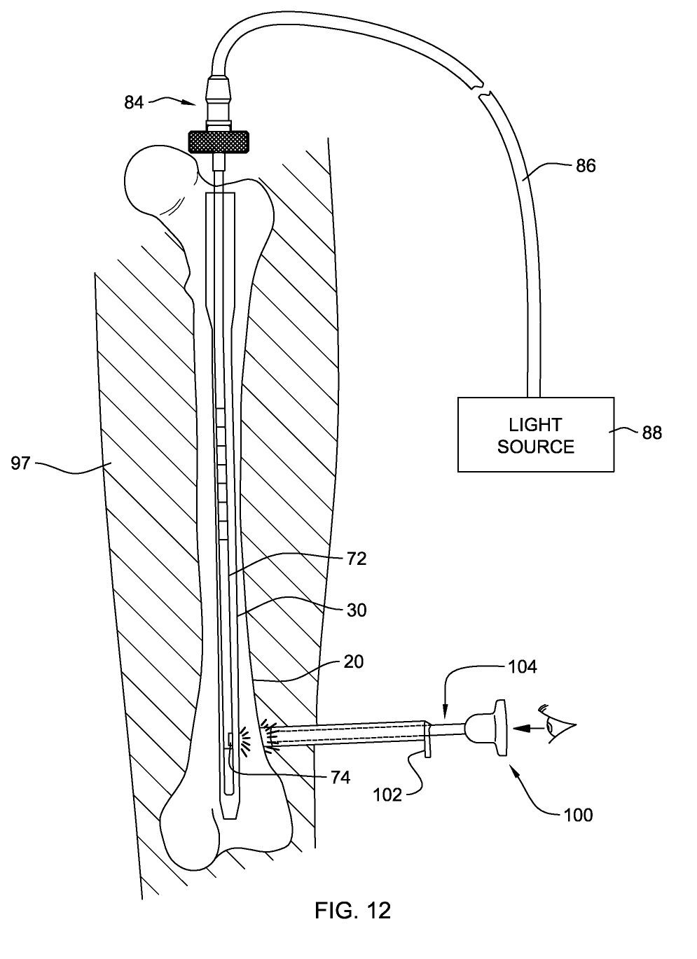

Referring to FIG. 12, the light emitted by the silhouette of the distal locking hole is observed by either direct vision, direct vision with arthroscope or arthroscopic established technique with an external camera 100 and monitor screen (not shown). The unique aspect of the use of the arthroscope in this case is that the light source is from inside the medullary canal and not emitted from the arthroscope itself. A primary cannula 102 and an arthroscope cannula 104 may be used.

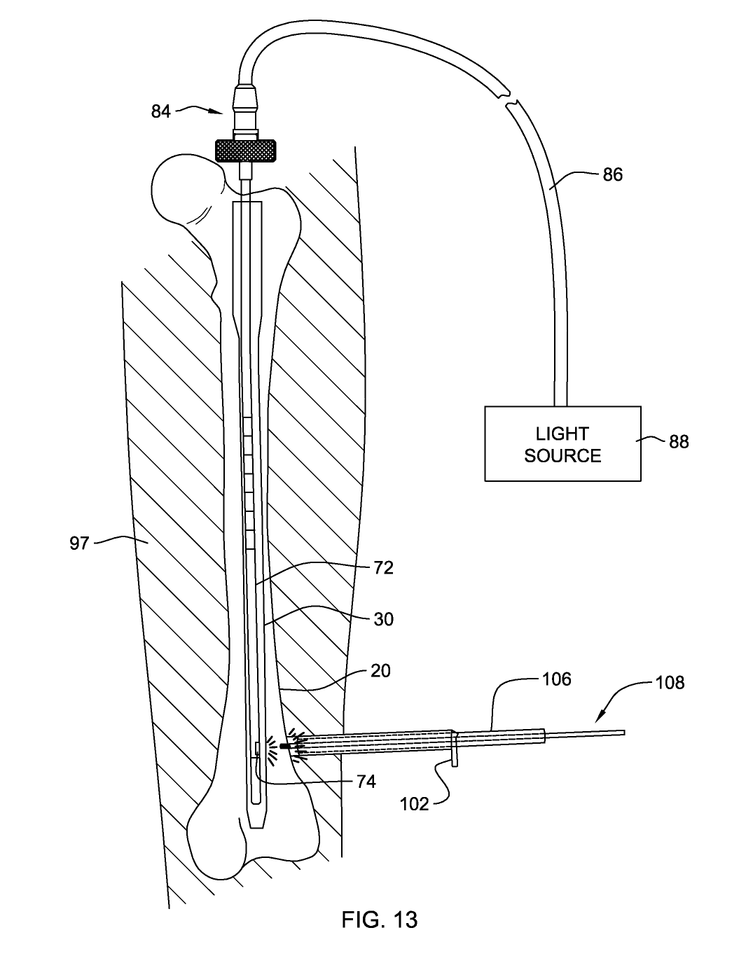

Referring to FIG. 13, once the transillumination target is acquired with either direct vision or with the arthroscope, the arthroscope cannula 104 is removed and a third or guide pin cannula 106 having an inner diameter matching a threaded guide wire 108, and an outer diameter matching the inside of the primary cannula 102 is inserted. The threaded guide pin 108 is then advance by hand or with a drill into the first outer cortex of the bone 20. Member 72 of the apparatus 70 for intramedullary transillumination is then removed for the facilitation of advancing the guide pin 108 all the way through the locking hole of the intramedullary rod 30.

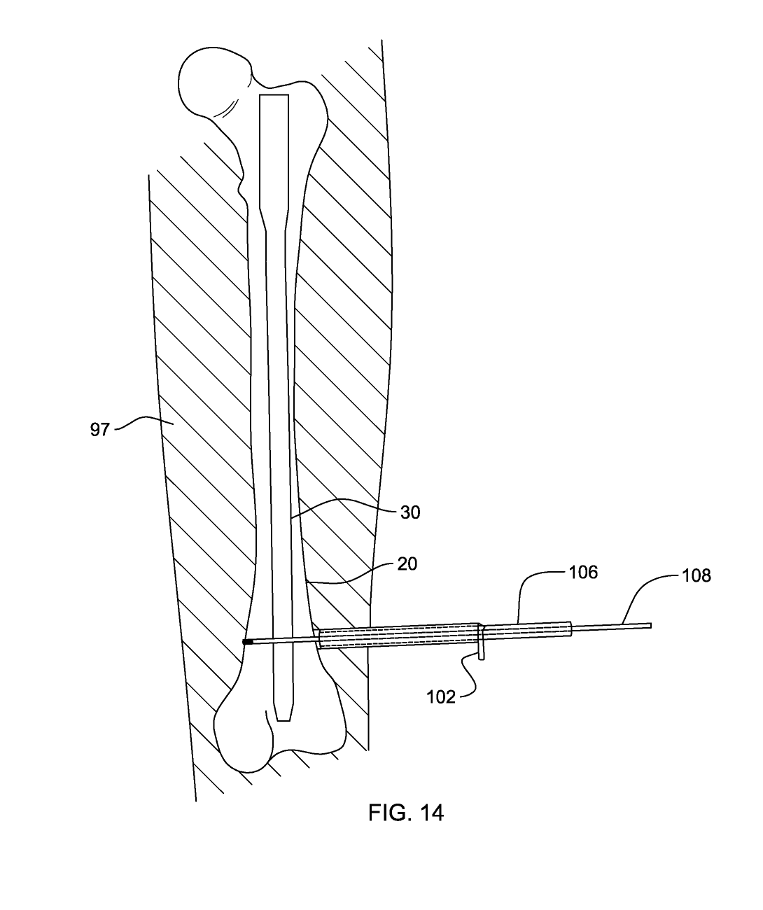

Referring to FIG. 14, member 72 of the apparatus 70 for intramedulary transillumination has been removed, the guide pin is advance to proper depth to just penetrate the far cortex. Established techniques are then used to verify the length of the screw measuring off the guide pin and cannula that are exposed out of the bone. This can be verified using x-ray or fluoroscopy.

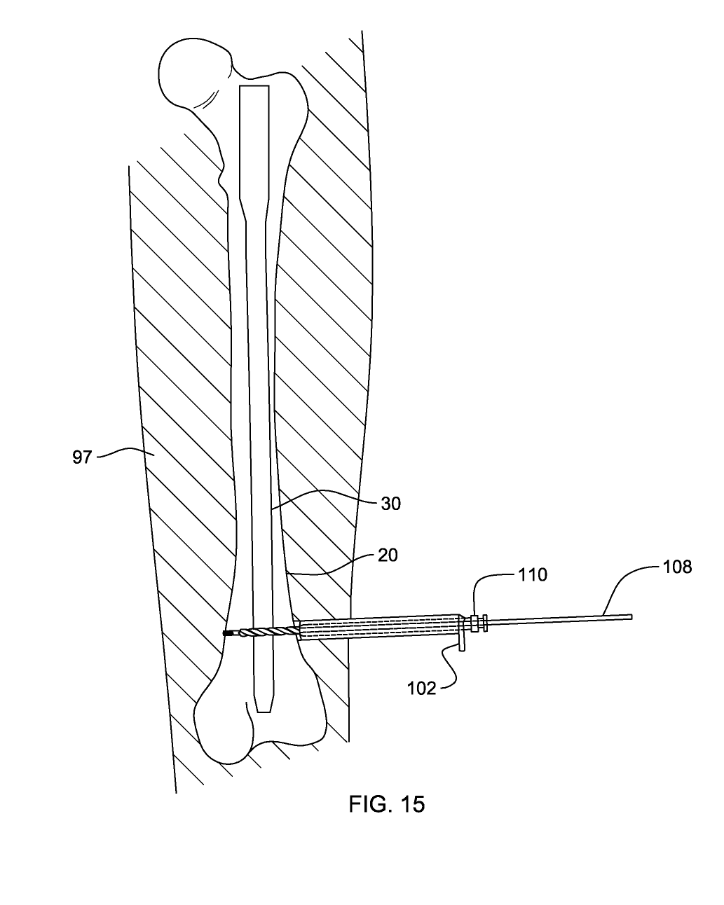

Referring to FIG. 15, once the guide pin 108 has been advanced to the proper depth a cannulated drill bit 110 is inserted over the guide pin 108 and advanced to the far cortex. Drill 110 can be calibrated to allow reading the depth directly off the drill and its most proximal portion on a cannula of predetermined or standard length.

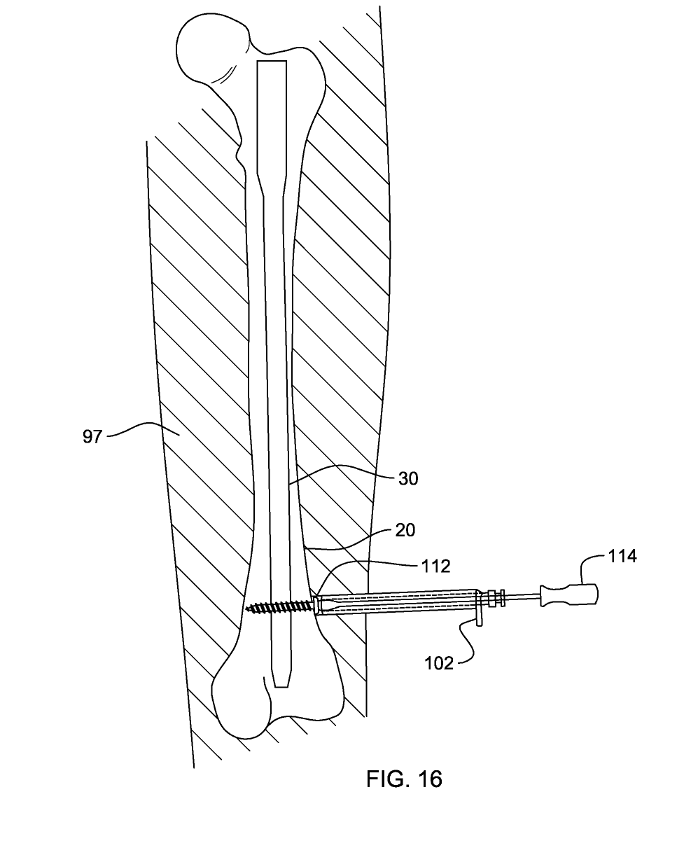

Referring to FIG. 16, following the completion of the drilling of the appropriate diameter hole for the corresponding locking screw 112, the primary cannula is held in place and the locking screw is inserted through the primary cannula, and placed with the aid of a screw driver 114, thus completing the procedure. The position of screw 112 can then be verified with x-ray or fluoroscopy.

It will be understood that in accordance with standard techniques, light source cable 86 is constructed of materials which permit it to be sterilized prior to use, and is generally reusable. However member 72 of apparatus 70, while also constructed of materials which permit it to be sterilized, is designed to be disposable after use in a surgical procedure for a single patient.

Various modifications of the invention are possible. Instead of using fiber optic components, an LED within member 72 of apparatus 70 may provide a source of radiation of appropriate wavelength. Further, a solid-state laser emitter may be used instead of an LED. Collimated light may also be provided by a laser within light source 88.

It will be understood that when the illumination apparatus has been inserted into the bone and aligned with a distal locking hole in the intramedullary nail or rod 30, prior to beginning the portion of the surgical procedure performed to expose the bone for directly observing the illumination on the outer surface of the bone, the surgeon may carefully observe the tissue over the bone that is being illuminated. In many cases, such careful observation and inspection will help make the surgeon aware of critical tissue structures, so that injury to such structures can be avoided or greatly minimized when exposing the bone, thus leading to a much more satisfactory recovery for the patient.

It should be understood that the foregoing description is only illustrative of the invention. Various alternatives and modifications can be devised by those skilled in the art without departing from the invention. Accordingly, the present invention is intended to embrace all such alternatives, modifications and variances.

What is claimed is:

1. Apparatus for use with a surgical drill in the repair of bones using an intramedullary nail insertable into a patient's bone, said intramedullary nail having a hollow body portion and a distal transverse hole, comprising, in combination:
a rod like device for insertion into the intramedullary nail, said device having a light source emitting electromagnetic non-ionizing radiation in the infrared or visible portions of the electromagnetic spectrum, and said device being positionable so that said light source emits said radiation through said distal transverse hole of said intramedullary nail; and
a surgical instrument for exposing an exterior surface of a portion of said bone illuminated by said radiation for view by the surgeon;
whereby detection of said radiation on an exterior surface of said bone and alignment of said surgical drill to said radiation passing through said transverse hole of said intramedullary nail aligns said surgical drill with said transverse hole, permitting accurate drilling of a hole through said bone and passage of the drill through the transverse hole of said intramedullary nail.

2. The apparatus of claim 1, wherein said surgical instrument comprises:
a primary cannula configured for extending from outside the patient to a location within the patient from which the portion of the bone illuminated by the radiation is visible; and
an arthroscope cannula for insertion into the primary cannula to allow the surgeon to view the exterior surface of the portion of the bone illuminated by said radiation.

3. The apparatus of claim 1, wherein said surgical instrument comprises:
a primary cannula configured for extending from outside the patient to a location within the patient from which the portion of the bone illuminated by the radiation is visible;
a guide pin cannula for insertion within said primary cannula; and
a threaded guide pin for insertion into said guide pin cannula, and for making an initial opening in the portion of the bone illuminated by the radiation.

4. The apparatus of claim 3, further comprising a cannulated drill having a cannula sized to accept said guide pin so that said drill is guided by said guide pin to drill said hole through the bone and to assist in aligning of the drill so that the drill passes through the transverse hole of said intramedullary nail, when said device is moved within said intramedullary nail so that said device is clear of the transverse hole, and said guide pin cannula has been removed from said primary cannula.

5. The apparatus of claim 1, wherein said surgical instrument comprises:
a screw driver for insertion into said primary cannula, for driving a screw into said bone and through the transverse hole of said intramedullary nail.

6. The apparatus of claim 1, wherein said surgical instrument comprises a scalpel for making an incision in tissues of the patient to permit the surgeon to directly view the exterior surface of a portion of said bone illuminated by the radiation.

7. The apparatus of claim 6, wherein said surgical instrument further comprises a retractor for retracting the tissues of the patient on at least one side of the incision, to assist the surgeon in obtaining the direct view of the exterior surface of a portion of said bone illuminated by the radiation.

8. The apparatus of claim 1, further comprising the surgical drill for drilling the hole through said bone and passing through the transverse hole of said intramedullary nail.

9. The apparatus of claim 1, wherein said light source comprises a laser.

10. The apparatus of claim 1, further comprising a shaping mask for said light source for providing a shape to a beam of radiation from said device, to assist in precisely locating a center of said beam.

11. A surgical kit for use with a surgical drill in the repair of bones using an intramedullary nail insertable into a patient's bone, said intramedullary nail having a hollow body portion and a distal transverse hole, said kit comprising:
a rod like device for insertion into the intramedullary nail, said device having a light source emitting electromagnetic non-ionizing radiation in the infrared or visible portions of the electromagnetic spectrum, and said device being positionable so that said light source emits said radiation through said distal transverse hole of said intramedullary nail; and
a surgical instrument for exposing an exterior surface of a portion of said bone illuminated by said radiation for view by the surgeon.

12. The surgical kit of claim 11, wherein said surgical instrument comprises:
a primary cannula configured for extending from outside the patient to a location within the patient; and
an arthroscope cannula for insertion into the primary cannula.

13. The surgical kit of claim 11 wherein said surgical instrument comprises:
a primary cannula configured for use for extending from outside the patient to a location within the patient;
a guide pin cannula for insertion within said primary cannula; and
a threaded guide pin for insertion into said guide pin cannula.

14. The surgical kit of claim 13, further comprising a cannulated drill having a cannula sized to accept said guide.

15. The surgical kit of claim 11, wherein said surgical instrument comprises:
a screw driver for insertion into said primary cannula, for driving a screw into said bone and through the transverse hole of said intramedullary nail.

16. The surgical kit of claim 11, wherein said surgical instrument comprises a scalpel for making an incision in tissues of the patient.

17. The surgical kit of claim 16, wherein said surgical instrument further comprises a retractor for retracting the tissues of the patient on at least one side of the incision.

18. The surgical kit of claim 11, further comprising the surgical drill for drilling the hole through said bone.

19. The surgical kit of claim 11, wherein said light source comprises a laser.

20. The surgical kit of claim 11, further comprising a shaping mask for said light source for providing a shape to a beam of radiation from said device, to assist in precisely locating a center of said beam.

21. A method for use with a surgical drill in the repair of bones using an intramedullary nail insertable into a patient's bone, said intramedullary nail having a hollow body portion and a distal transverse hole, comprising:
inserting a rod like device into the intramedullary nail, said device having a light source emitting electromagnetic non-ionizing radiation in the infrared or visible portions of the electromagnetic spectrum, and said device being positionable so that said light source emits said radiation through said distal transverse hole of said intramedullary nail; and
exposing an exterior surface of a portion of said bone illuminated by said radiation for view by the surgeon;
detecting said radiation on the exterior surface of said bone;
aligning said surgical drill to said radiation passing through said transverse hole of said intramedullary nail;
drilling of a hole through said bone and passage of the drill through the transverse hole of said intramedullary nail.
